(19) United States
(12) Patent Application Publication
(10) Pub. No.: US 2013/0023820 A1
(43) Pub. Date: Jan. 24, 2013

(54) PATIENT-CONTROLLED ANALGESIA SAFETY SYSTEM

(71) Applicants: CLIFFORD T. SOLOMON, Severna Park, MD (US); THEODORE C. SOLOMON, Hampstead, MD (US)

(72) Inventors: CLIFFORD T. SOLOMON, Severna Park, MD (US); THEODORE C. SOLOMON, Hampstead, MD (US)

(21) Appl. No.: 13/556,147

(22) Filed: Jul. 23, 2012

Related U.S. Application Data
(60) Provisional application No. 61/510,400, filed on Jul. 21, 2011.

(51) Int. Cl. A61M 5/168

(52) U.S. Cl. 604/66

(57) ABSTRACT

The patient-controlled analgesia (PCA) safety system includes a housing with a hollow compartment. A display and keypad are disposed on one side of the for display of monitoring data and selective commands respectively. An analgesic module and an antidote module are detachably mounted inside the compartment. A control module is disposed inside the housing. A vital signs monitor is connected to the control module to monitor a patient. In the event of abnormal readings due to, e.g., overdose or drug-related complications, the control module can automatically administer an antidote to the analgesia. The control module can also alert a nurse monitor and/or other personnel wirelessly to insure someone is notified of the potential emergency. The PCA safety system can also be provided in a separate safety module having a detachably mounted antidote module.

### CROSS-REFERENCE TO RELATED APPLICATION

This application claims the benefit of U.S. Provisional Patent Application Ser. No. 61/510,400, filed Jul. 21, 2011.

### BACKGROUND OF THE INVENTION

1. Field of the Invention

The present invention relates to medical systems, and particularly to a patient-controlled analgesia (PCA) safety system that automatically prevents potential adverse effects of the analgesic or the unintended misuse or overdose thereof.

2. Description of the Related Art

Postoperative patients generally suffer a great deal of pain as a result of the surgical procedure. The physician usually prescribes strong analgesics or painkillers in the form of pills or tablets to relieve this symptom for most patients. For some extreme cases, more powerful analgesics are prescribed which can be self-administered by the patient through a PCA device or pump. The typical PCA device is programmed to deliver a predetermined amount of opiate, such as morphine, through a connected intravenous tube whenever the patient presses a button according to the patient's perceived needs. While the PCA device relieves pressure on nursing resources and grants superior pain control, the patient could easily induce an accidental overdose if the administration of the analgesic is left unchecked or if the patient is hypersensitive to the analgesic. Most PCA devices include some type of safety system that helps prevent such dangers, an example of which is a time delay, either programmed or built into the device. The time delay prevents the device from processing another dose before the specified delay has lapsed.

Despite these safeguards, accidental overdoses can still occur, and the typical PCA device still lacks a more active approach in preventing the overdose or other drug related complications. Like most devices, a PCA pump is prone to operator error. For example, a nurse who prepares the PCA device for patient use may inadvertently install a cartridge or vial that contains a higher concentration of analgesic than prescribed, e.g., a 5 mg/mL versus a 1 mg/mL. This can lead to an overdose, even on the first charge. Moreover, the physiology of everyone is not the same. A certain dosage for one may not result in the same pain relief for another. Some may even be allergic to certain drugs and/or concentrations thereof without prior knowledge to themselves or to the physician. Whatever complications may arise, a typical PCA device does not have the means to overcome these types of issues with any alacrity. Failure to respond timely in those situations can ultimately lead to the death of the patient.

In light of the above, it would be a benefit in the medical arts to provide a self-administering drug delivery system with safety features capable of preventing overdose and responding to other emergencies in a timely manner. Thus, a patient-controlled analgesia safety system solving the aforementioned problems is desired.

### SUMMARY OF THE INVENTION

The patient controlled analgesia safety system includes a housing with a hollow compartment. A display and keypad are disposed on one side of the housing for display of monitoring data and selective commands respectively. An analgesic module and an antidote module are detachably mounted inside the compartment. A control module is disposed inside the housing. A vital signs monitor is connected to the control module to monitor a patient. In the event of abnormal readings due to, e.g., overdose or drug related complications, the control module can automatically administer an antidote to the analgesia. The control module can also alert a nurse monitor and/or other personnel wirelessly to insure someone is notified of the potential emergency. The PCA safety system can also be provided in a separate safety module having a detachably mounted antidote module.

These and other features of the present invention will become readily apparent upon further review of the following specification and drawings.

### BRIEF DESCRIPTION OF THE DRAWINGS

FIG. 1 is an environmental, perspective view of a patient-controlled analgesia (PCA) safety system according to the present invention.

FIG. 2 is a schematic diagram of the controls in the PCA safety system according to the present invention.

FIG. 3 is a perspective view of an alternative embodiment of a PCA safety system according to the present invention.

Similar reference characters denote corresponding features consistently throughout the attached drawings.

### DETAILED DESCRIPTION OF THE PREFERRED EMBODIMENTS

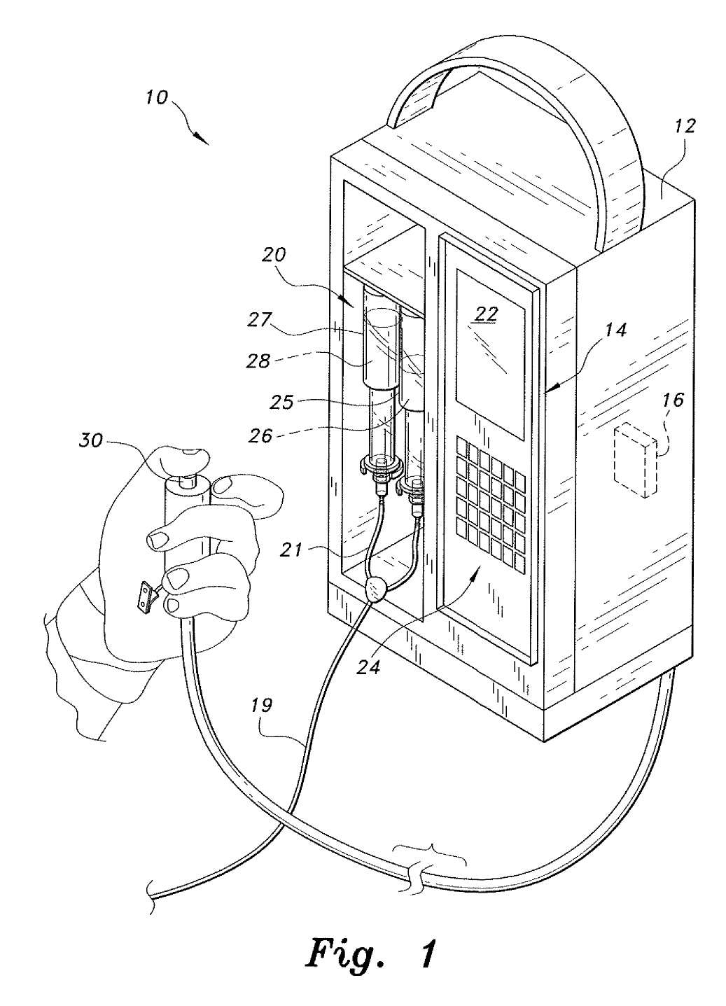

The patient-controlled analgesia (PCA) safety system, generally referred to by the reference number 10 in the drawings, includes a variety of features for timely response to many drug related emergency situations, both in hospitals and at home. As shown in FIG. 1, the PCA safety system 10 includes a dispenser 12, a control panel 14, a control module 16, an analgesic module 18 and an antidote module 20.

The control panel 14 includes a keypad 24 and a display monitor 22. The control panel 14 allows the user to input various commands and program the device. For example, depending on the prescription, the nurse or physician may set the minimum time intervals before another dose of analgesic can be self-administered by the patient. Other parameters that can be adjusted include the rate in which certain physical conditions are being monitored, the number of doses allowed prior to complete lockout of further doses for a predetermined period of time, the name and other personal data of the patient, and the name of the physician in charge of the patient. All or some of that information can be displayed on the monitor 22. The monitor 22 can be a color or monochromatic LCD (liquid crystal display) capable of displaying the above information. As a non-limiting alternative, the keypad 24 may be a touchpad or an alphanumeric pad incorporated into the monitor 22 as a graphical interface accessible via a touchscreen.

The analgesic module 18 disposed in the dispenser 12 can be provided in the form of a cartridge 25. The cartridge 25 includes a vial, ampoule, canister or container 26 filled with the prescribed analgesic. A first or primary intravenous line 19 connects the contents of the vial 26 to the user or patient allowing for selective delivery of the analgesic whenever the user or patient activates the dispenser 12 via the patient-controlled actuator 30, usually a hand-held control with a button. The cartridge 25 allows for more easy preparation and installation of the drug.

One of the many safety features of the PCA safety system 10 lies in the antidote module 20. In this non-limiting exemplary embodiment, the antidote module 20 can be provided in the form of an antidote cartridge 27 detachably mounted in the dispenser 12 in the same manner as the analgesic cartridge 25. The antidote cartridge 27 includes a vial, ampoule, canister or container 28 tilled with an antidote for the analgesic contained in the analgesic vial 26. The antidote can be Naloxone™, an opioid antagonist or any other drug or chemical compound that counteracts the effects of the analgesic in the event of an overdose. A second intravenous line 21 connects the contents of the antidote vial 28 to the first intravenous line 19 to facilitate delivery of the antidote.

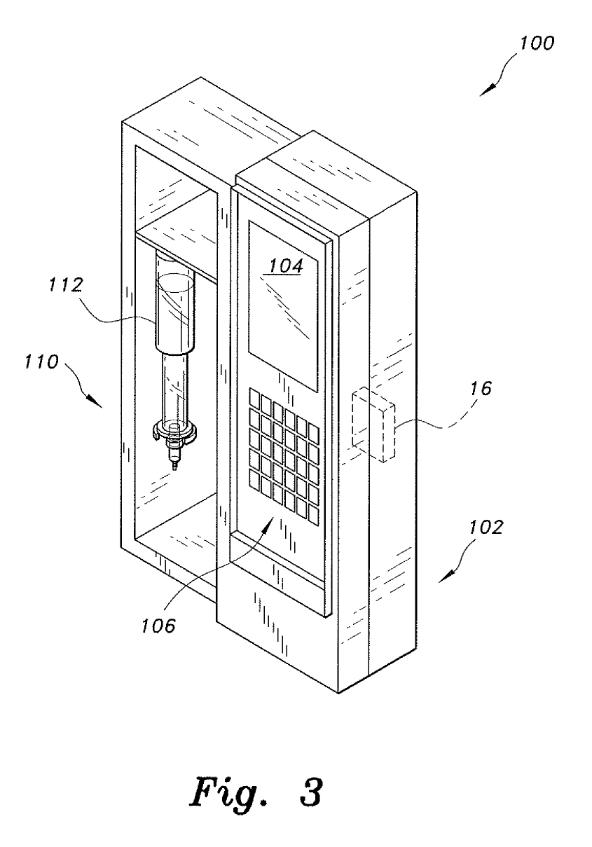

The operation and medical alerts of the PCA safety system 10 are controlled by the control module 16. As schematically shown in FIG. 3, the control module 16 includes a vital signs monitor 40 connected to a central processor 50. In the non-limiting exemplary embodiment, the vital signs monitor 40 is preferably connected to the monitoring systems already in place within the healthcare facility. For use in home settings or healthcare facilities with limited resources, the vital signs monitor 40 can include various sensors built-in or integrated into the vital signs monitor 40 so as to provide the necessary medical parameters data for evaluating and determining an emergency situation such as an overdose. These sensors can include, but not limited to, a blood pressure sensor 42, a pulse rate sensor 44, an oxygen/CO2 sensor 46, and a respiration sensor 48. The data from the sensors can be stored in memory 52 to be accessed by the processor 50 and/or transmitted by the wireless transmitter 54.

In the event of abnormal readings from the vital signs monitor 40 indicative of potential analgesic or drug related complications, the PCA safety system 10 is preferably programmed so that the processor 50 discontinues any further attempts to self-administer the analgesic. If abnormal readings persist after a brief, predetermined interval, e.g., 30 seconds or less, then the processor 50 automatically transmits an alert to the nurse monitor 60 through the wireless transmitter 54 along with data from the vital signs monitor 40. At the same time, the processor 50 automatically activates the antidote module 20 to deliver the antidote to the patient.

The above may be sufficient to counteract the immediate danger and stabilize the patient. However, the patient must be examined by professionals to determine the extent of the emergency. To insure that someone is notified, the PCA safety system 10 includes several redundant safety features. One of the redundant features is in who receives the wireless transmission. If for some reason the nurse at the nurse monitor 60 is not able to respond timely to the alert, the processor can be programmed to transmit the alert to other personnel 62 such as an emergency response, the physician in charge, and family members in a selected priority list. In this manner, even if the healthcare professionals on site and primarily responsible do not respond immediately or timely, the physician or one of the patient's family members can notify someone at the patient's location of the emergency.

Another redundancy lies in safeguards against system component failure. In the event the wireless transmitter malfunctions or fails, a backup transmitter 56 is activated by the processor 50. In the non-limiting exemplary embodiment, the backup transmitter 50 is preferably capable of both landline and wireless transmissions for insuring the transmission reaches the desired recipients or receivers. A backup power source 58 is provided as a countermeasure against inadvertent power failure. The backup power source 58 supplies power to the processor 50 and/or the backup transmitter 56 for continuous functioning of the control module 16. The backup power source 58 is preferably a rechargeable and re-useable battery.

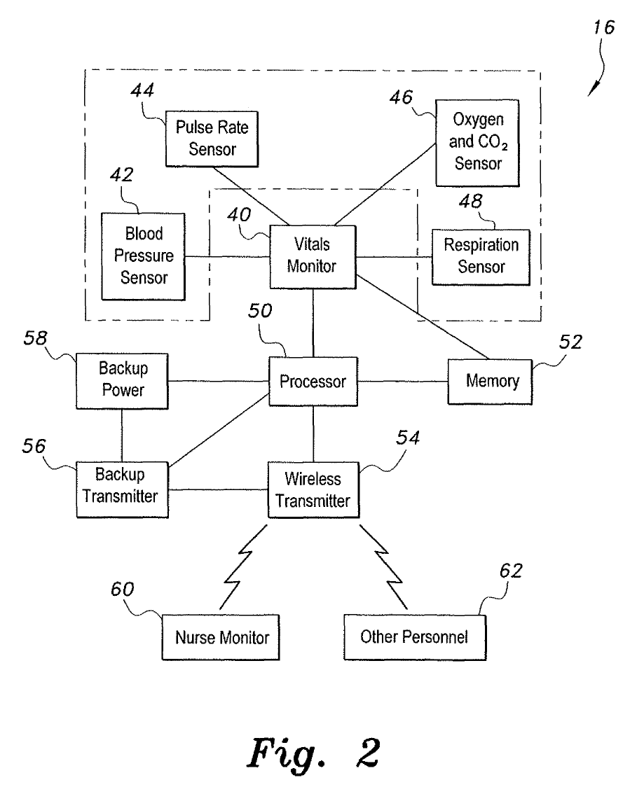

While the above describes the PCA safety system 10 as a PCA device with integrated safety features, the PCA safety system 10 may also be provided in a modular system. As shown in the non-limiting alternative embodiment in FIG. 2, the PCA safety system 100 includes a safety module 102 that can be attached to existing PCA devices by any attachment means such as hook-loop fasteners, straps, belts and other adjustable connectors. The safety module 102 can include a keypad 106 and a display monitor 104 similar to the ones discussed above. The keypad 106 facilitates input of various commands or programming of the safety module 102, and the display monitor 104 shows any desired and relevant data. As discussed above, the display monitor 104 may be a touchscreen that can replace the keypad 106 for input or be a means for inputting other on-screen functions.

The safety module 102 includes the control module 16 described above, the control module 16 being incorporated into the body of the safety module 102. The medical parameters of the patient can be obtained from existing monitoring equipment or from the integrated sensors of the control module. Once attached to an existing PCA device, the safety module 102 can be connected to a data port on the PCA device such that the safety module 102 can override, at the very least, further administration of the analgesic and/or power of the PCA device.

The safety module 102 can be used alone to help monitor the patient's reaction to the analgesia and facilitate notification of any drug related complications to the proper authorities or family. However, to be more effective, the PCA safety system 100 can include an antidote module 110 detachably mounted to the safety module 102. The antidote module 110 includes an antidote contained in a vial, ampoule or canister 112. The antidote is selectively and automatically administered by the processor 50 in response to the potential emergency conditions noted above.

Thus, it can be seen that the PCA safety system 10, 100 includes many safety features that can overcome a majority of analgesic or drug related complications. The antidote module 20, 110 provides an immediate countermeasure to a potential overdose, The control module 50 and the many redundant features insure that someone is notified of the patient's condition. Moreover, the various configurations allows the PCA safety system 10, 100 to be utilized in a wide range of facilities from a home setting to a state of the art hospital.

It is to be understood that the PCA safety system 10, 100 encompasses a wide variety of alternatives. For example, the PCA safety system 10, 100 is not limited to patient-controlled analgesia devices. The teachings thereof can be applied to any self-controlled or self-administered drug delivery systems such as an automated insulin pump and the like.

It is to be understood that the present invention is not limited to the embodiments described above, but encompasses any and all embodiments within the scope of the following claims.

We claim:

1. A patient-controlled analgesia (PCA) safety system, comprising:
a housing;
an antidote module detachably mounted to the housing, the antidote module having an antidote for a drug; and
a control module disposed inside the housing, the control module having means for obtaining medical parameters data of a subject and means for signaling medical professionals and family of potential drug-related complications;
wherein the control module automatically administers the antidote based upon abnormal medical parameters data indicative of drug-related complications and automatically to notifies medical professionals and family of the potential drug-related complications.

2. The PCA system according to claim 1, wherein said means for obtaining medical parameters data of a subject comprises a vital signs monitor and a programmable processor, said vital signs monitor capable of being connected to an existing monitoring system in order to obtain the data.

3. The PCA system according to claim 2, wherein said vital signs monitor further comprises a plurality of sensors operatively connected to said processor.

4. The PCA system according to claim 3, wherein said plurality of sensors comprises a blood pressure sensor, a pulse rate sensor, an oxygen/CO₂ sensor, and a respiration sensor.

5. The PCA system according to claim 2, wherein said means for signaling medical professionals and family of potential drug-related complications comprises a wireless transmitter connected to said processor.

6. The PCA system according to claim 5, wherein said means for signaling medical professionals and family of potential drug related complications further comprises a backup transmitter operatively connected to said wireless transmitter and said processor, said backup transmitter being configured for activation upon malfunction of said wireless transmitter,

7. The PCA system according to claim 5, further comprising a backup power source connected to said control module in order to provide emergency power.

8. A patient-controlled analgesia (PCA) device, comprising:
a housing having a hollow compartment;
means for inputting commands disposed on a side of the housing;
means for displaying data and functions adjacent the means for inputting commands;
an analgesic module detachably mounted in the compartment, the analgesic module having analgesia for selective delivery by a user;
an antidote module detachably mounted in the compartment, the antidote module having an antidote for the analgesia;
an actuator connected to the housing, the actuator adapted for being selectively actuated by the user for administering discreet amounts of the analgesia; and
a control module disposed inside the housing, the control module having means for obtaining medical parameters data of the user and a means for signaling medical professionals and family of potential drug related complications, the means for inputting and the means for displaying being connected to the control module;
wherein the control module automatically administers the antidote based upon abnormal medical parameters data indicative of drug-related complications and automatically notifies medical professionals and family of the potential drug-related complications.

9. The PCA device according to claim 8, wherein said means for inputting commands comprises a keypad.

10. The PCA device according to claim 8, wherein said means for displaying data and functions comprises an LCD screen.

11. The PCA device according to claim 8, wherein said means for inputting commands and said means for displaying data and functions comprises a touchscreen having a graphic interface for displaying input icons, data and functions.

12. The PCA system according to claim 8, wherein said means for obtaining medical parameters data of a subject comprises a vital signs monitor and a programmable processor, said vital signs monitor adapted for connection to an existing monitoring system in order to obtain the data.

13. The PCA system according to claim 12, wherein said vital signs monitor further comprises a plurality of sensors operatively connected to said processor.

14. The PCA system according to claim 13, wherein said plurality of sensors comprises a blood pressure sensor, a pulse rate sensor, an oxygen/CO₂ sensor, and a respiration sensor.

15. The PCA system according to claim 12, wherein said means for signaling medical professionals and family of potential drug-related complications comprises a wireless transmitter connected to said processor.

16. The PCA system according to claim 15, wherein said means for signaling medical professionals and family of potential drug-related complications further comprises a backup transmitter operatively connected to said wireless transmitter and said processor, said backup transmitter configured for activation upon malfunction of said wireless transmitter.

17. The PCA system according to claim 15, further comprising a backup power source connected to said control module in order to provide emergency power.
